(19) United States
(12) Patent Application Publication
(10) Pub. No.: US 2016/0331598 A1
(43) Pub. Date: Nov. 17, 2016

(54) ABSORBENT ARTICLE WITH GRAPHIC FRAMING

(71) Applicant: The Procter & Gamble Company, Cincinnati, OH (US)

(72) Inventors: Kelly Lynn Morrison, South Lebanon, OH (US); Brittany Canfield, Cincinnati, OH (US)

(21) Appl. No.: 15/153,880

(22) Filed: May 13, 2016

Related U.S. Application Data
(60) Provisional application No. 62/161,666, filed on May 14, 2015.

(51) Int. Cl. A61F 13/47; A61F 13/84

(52) U.S. Cl. CPC A61F 13/47; A61F 13/84; A61F 2013/8497

(57) ABSTRACT

The present application relates to absorbent articles, including catamenial devices, such as tampons and sanitary napkins, for the absorption of menses. More particularly, the present invention relates to an absorbent article having a product graphic framing arrangement that frames the central usage area of the absorbent core.

### FIELD OF INVENTION

The present application relates to absorbent articles, including catamenial devices, such as sanitary napkins, for the absorption of menses. More particularly, the present invention relates to catamenial devices comprising one or more graphical images wherein the graphical images outline the functional area of the absorbent article.

### BACKGROUND OF THE INVENTION

Disposable absorbent articles, such as sanitary napkins, diapers, adult incontinence, and catamenial pads are manufactured at a high rate of speed. However today's modern high speed production lines have an inherent variation in the cross direction (CD) tracking of webs and the combining of webs. If noticed by the consumer, this CD variation can impact the quality perception of the product. For example, a consumer may believe that a product will not work properly if they notice that the absorbent core is not centrally located between the wing components-of the article. Further, today's modern high speed production lines may include variation in the machine direction (MD).

Regarding the core of the absorbent article, the functional area of the core is often larger than the absorbency need of the consumer. Non-identical cores allow for improvements in high speed processing without sacrificing the ability to exceed the consumer need. However, a consumer may perceive intentional differences between cores as a flaw in a product.

In addition, consumers like color. Color is often added in the form of graphics or graphical elements to a layer in the product. Printing graphical elements on the top layer of the article can communicate a barrier signal or highlight the functional area of the product. However, printed product graphical elements, like barrier signals to highlight the functional area of the product, that are consumer preferred can actually accentuate the perception of CD variation found in today's products, making the consumers believe that the article is defective, when in fact the product is still within quality standards to function properly. To deal with this problem, current products either sacrifice an effective barrier signal graphic or are forced to incur large expenses to slow down the lines or purchase specialized equipment to reduce the CD variation. These costs can be prohibitive.

For example, current products have printed far away from the perimeter of the core so that the core placement perception will be more acceptable for the large CD variation. Graphical elements have also been placed on the entire backsheet before the placement of the core to not have to be concerned with the CD variation. Lastly, graphical elements have been placed in an asymmetric fashion in areas outside of the core perimeter to avoid dealing with the CD variation.

As such, what is needed is a specific printed graphic orientation visible from the body side surface of an article that enables a consumer acceptable signal and is capable of being created in a high speed manufacturing process. Additionally, there is a need to create a printed graphic orientation that highlights the functional area of the core in non-identical cores.

### SUMMARY OF THE INVENTION

An absorbent article having a liquid pervious topsheet, a backsheet joined to the topsheet, and an absorbent core having a perimeter disposed between the topsheet and the backsheet. The topsheet has an inner surface oriented toward the interior of the absorbent article or garment facing side and an outer surface oriented toward the skin of the wearer when the absorbent article is being worn or body facing side. The backsheet has an inner surface oriented toward the interior of the absorbent article and an outer surface oriented toward the garment of the wearer when the absorbent article is being worn. The absorbent core has an inner surface oriented toward the skin of the wearer when the absorbent article is being worn and an outer surface oriented toward the garment of the wearer when the absorbent article is being worn. The topsheet has one or more graphical elements forming a product graphic framing arrangement. The product graphic framing arrangement overlap the perimeter of the absorbent core such that between 70% and 95% of the absorbent core lies within the graphical elements.

### BRIEF DESCRIPTION OF THE DRAWINGS

While the specification concludes with claims particularly pointing out and distinctly claiming the subject matter of the present invention, it is believed that the invention may be more readily understood from the following description taken in connection with the accompanying drawings, in which:

FIG. 1 is a top view of a representative absorbent article.

FIG. 2A-C is a top view of absorbent articles having a product graphic framing arrangement.

FIGS. 3 is a top view of absorbent article having a product graphic framing arrangement.

FIGS. 4A-4E is a top view of absorbent articles having a product graphic framing arrangement including graphical elements.

FIGS. 5A-5B is a top view of absorbent articles having a product graphic framing arrangement.

### DETAILED DESCRIPTION OF THE INVENTION

The present invention is directed to printed product graphics and to absorbent articles, particularly disposable absorbent articles, having one or more graphics visible from the body facing side of the topsheet. The product graphic framing arrangement allows for high speed manufacturing while highlighting the functional zone of the article. The product graphic framing arrangement allows for the use of non-identical cores. Disposable absorbent articles can be feminine hygiene articles, including incontinence devices and catamenial products, such as, sanitary napkins, pantiliners, interlabial products, and the like. For convenience, the invention is disclosed below with respect to the embodiment of a catamenial device, such as a sanitary napkin or pantiliner.

As used herein, a “functional layer signal” relates to any identifier that draws attention to the functional absorbent portions of the absorbent article. The functional layer signal can be a structural identifier such as, for example, apertures in the form of slots or dots in or around the functional layer, graphical elements on the viewing portion of the functional layer or around the functional layer, or a combination of structural elements and graphical elements that direct a user to the functional layer of the absorbent article.

As used herein, “non-identical” cores relates to absorbent cores that have a surface area within 90% to 110% of a target surface area. The non-identical cores have a uniform central usage area that is at least 70% of the absorbent core total functional area.

As used herein, a “printed graphic orientation” relates to a set of graphical elements within a given area. However, as mentioned, the graphical elements need not be printed on the same surface within the area. The printed graphic orientation may outline the functional area of the absorbent core.

As used herein, a “product graphic framing arrangement” relates to the use of one or more graphics to frame the central usage area of the absorbent core. The product graphic framing arrangement may be a combination of graphical elements visible from the body side surface of the absorbent article. The product graphic framing arrangement may be printed on any layer visible from the body side of the product provided that at least a portion of the product graphic framing arrangement is printed on the absorbent core or a layer between the absorbent core and the body side facing uppermost layer of the product or the bodyside layer, such as for example, the absorbent core, a secondary topsheet, an additional acquisition layer, a topsheet, or any combinations thereof. The product graphic framing arrangement frames between 80% and 100% of the perimeter of the absorbent core, such as, for example, between 82% and 98%, 85% and 95%, 88% and 90%. The product graphic framing arrangement may be printed on either the upper or the lower surface of any of the layers provided it is visible from the body facing side. The product graphic framing arrangement can have one or more graphical elements. The product graphic framing arrangement serves as a functional layer signal. The product graphic framing arrangement may extend from the central usage area of the absorbent core towards the edges of the product. The product graphic framing arrangement may extend from the central usage area to the edge of the product for any given distance between the central usage area to an edge, such as, between 5% and 100% of the distance, such as, for example, 10%, 15%, 20%, 25%, 30%, 35%, 40%, 45%, 50%, 55%, 60%, 65%, 70%, 75%, 80%, 85%, 90%, or 95% of the distance between the central usage area and the product edge. The product graphic framing arrangement further allows for the use of non-identical cores.

Regardless of the particular configuration of the layers used in forming the absorbent article, a plurality of graphical elements may be printed in addition to a product graphic framing arrangement so that they are visible from the body-facing surface creating an overall product graphic arrangement. The particular graphical elements are selected to provide a unique visual appearance that highlights the functional zone to the consumer while allowing for high speed manufacturing of the absorbent article. The product graphic framing arrangement may be used in conjunction with non-identical cores. High speed manufacturing is done by placing one layer of material on top of another layer until the article is complete. Each layer of material must be placed within a calculated margin of error. Otherwise, the manufacturing system can deem that the product is unacceptable. Although a product can be acceptable due to the manufacturing guidelines, it is possible to have an absorbent article wherein the absorbent core has shifted in the CD direction along the transverse axis, the MD direction, or both. The inventors have found that by placing the graphical elements in a unique printed graphic orientation, the absorbent article can have consumer acceptable graphical elements that highlight the functional zone while allowing for high speed manufacturing.

The inventors have also found that the use of a product graphic framing arrangement allows for the highlighting of the central usage area of the absorbent core. Highlighting the central usage area of the absorbent core allows for the use of non-identical cores without impacting consumer acceptability. The non-identical cores may be placed in a package together for consumer use.

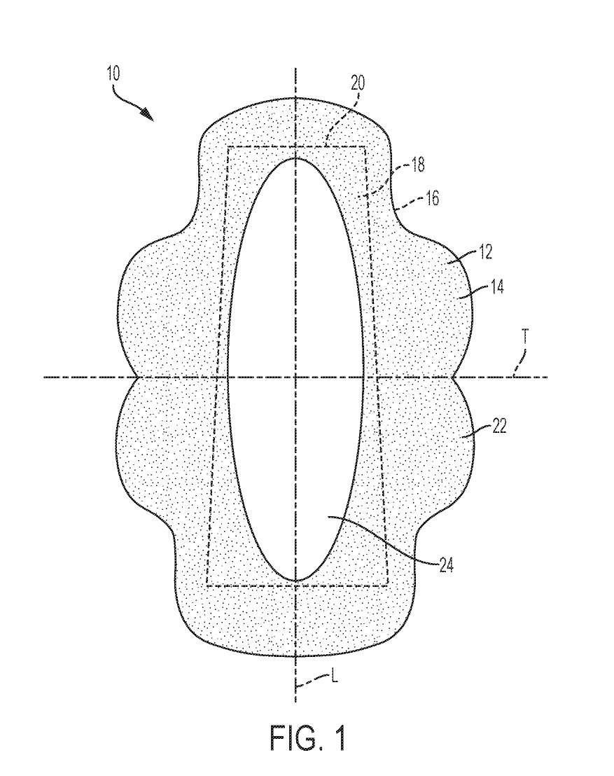

FIG. 1 shows an absorbent article 10, e.g., having a body-contacting surface 12 comprising an outer surface of a topsheet 14, a liquid impervious backsheet 16 joined to the topsheet 14, and an absorbent core 18. The absorbent article 10 has a longitudinal axis L, a transverse axis T. The longitudinal axis and the transverse axis meet at a central point of the sanitary napkin. The absorbent core has a perimeter 20.

As shown in FIG. 1, the absorbent article has a product graphic framing arrangement 22 that frames the central usage area 24 of the absorbent core 18.

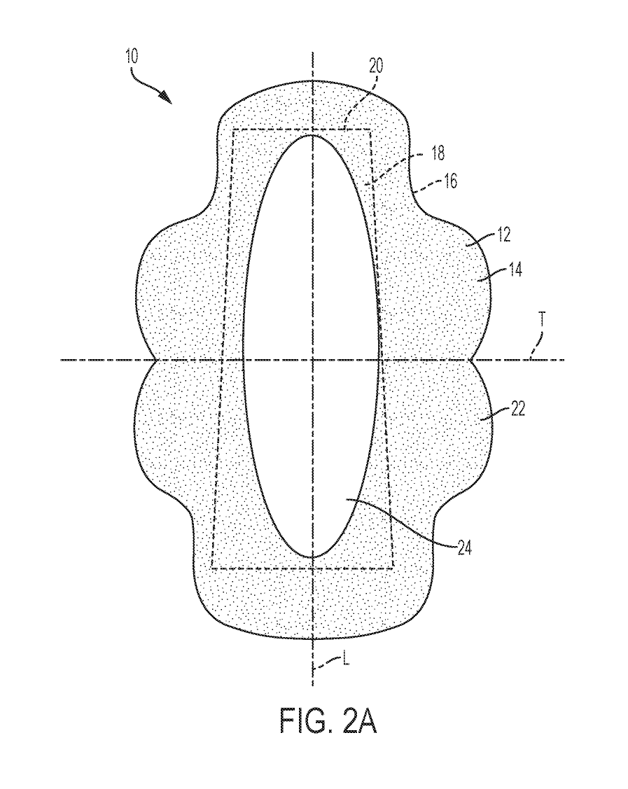

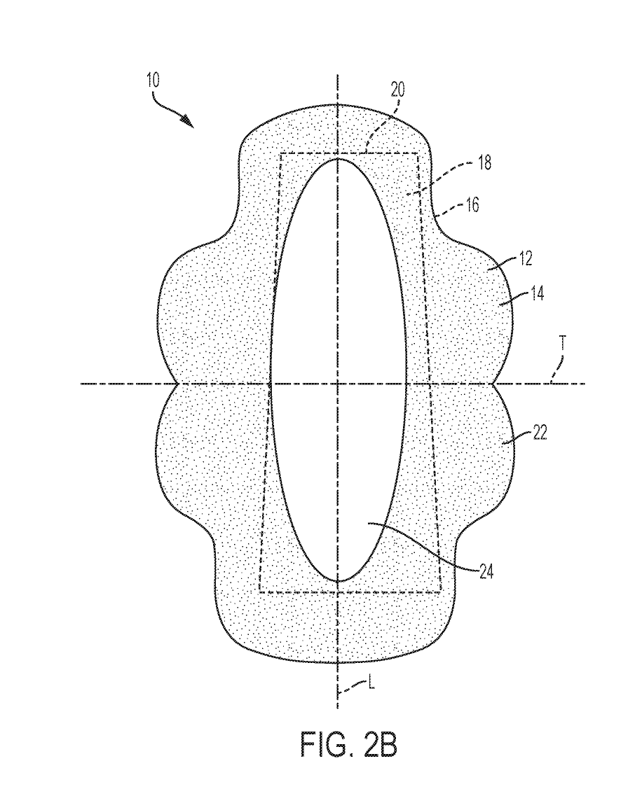

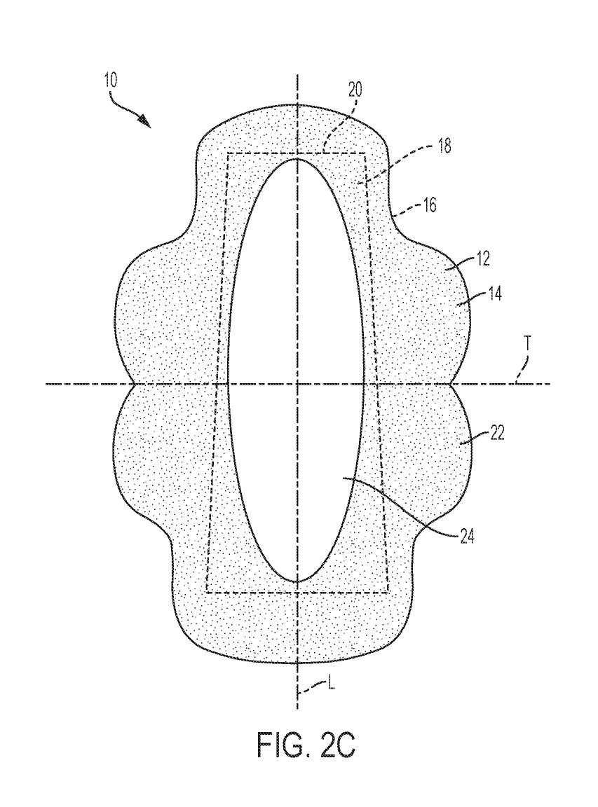

FIG. 2A-C show an absorbent article 10, e.g., having a body-contacting surface 12 comprising an outer surface of a topsheet 14, a liquid impervious backsheet 16 joined to the topsheet 14, and an absorbent core 18. The absorbent article 10 has a longitudinal axis L, a transverse axis T. The longitudinal axis and the transverse axis meet at a central point of the sanitary napkin. As shown in FIG. 2A and 2B, the absorbent core 18 may be shifted along the CD direction such that the absorbent core 18 is not central to the longitudinal axis. As shown in FIG. 2C, the absorbent core 18 may be shifted along the MD direction. The product graphic framing arrangement 22 frames the central usage area 24 of the absorbent core 18 for FIG. 2A, FIG. 2B, and FIG. 2C. As stated above, the use of the product graphic framing arrangement 22 allows for variability along the CD and MD directions created by high speed manufacturing while maintaining consumer acceptability.

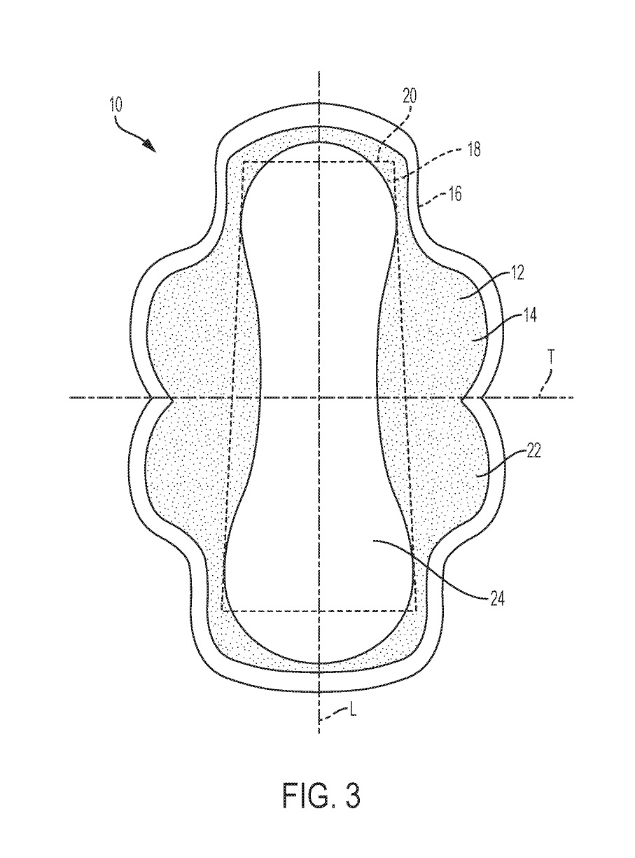

FIG. 3 show an absorbent article 10, e.g., having a body-contacting surface 12 comprising an outer surface of a topsheet 14, a liquid impervious backsheet 16 joined to the topsheet 14, and an absorbent core 18. The absorbent article 10 has a longitudinal axis L, a transverse axis T. The longitudinal axis and the transverse axis meet at a central point of the sanitary napkin. The product graphic framing arrangement 22 frames the central usage area 24 of the absorbent core 18. As shown in FIG. 3, the product graphic framing arrangement does not extend all the way out to the edge of the absorbent article 10.

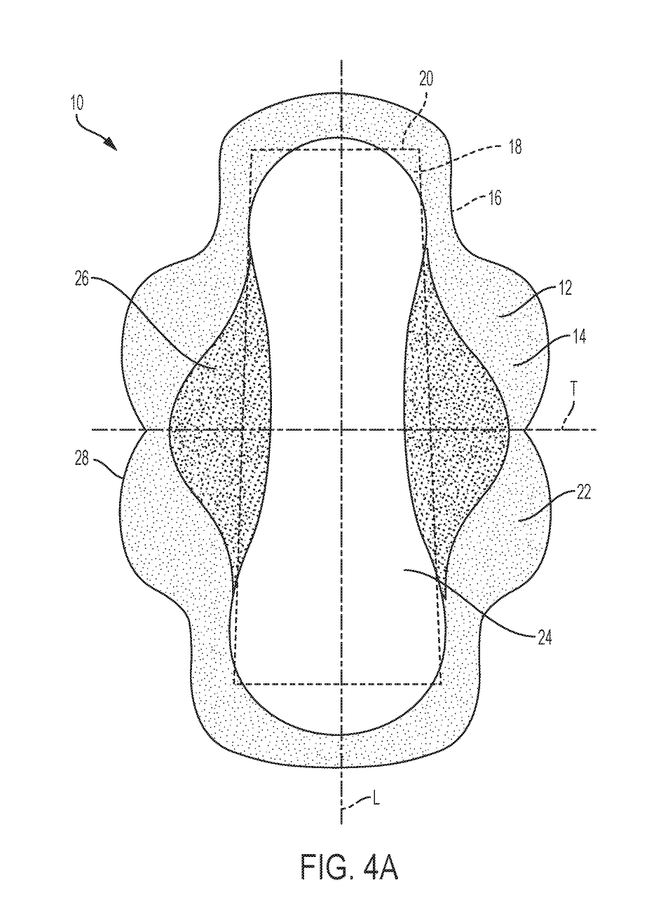

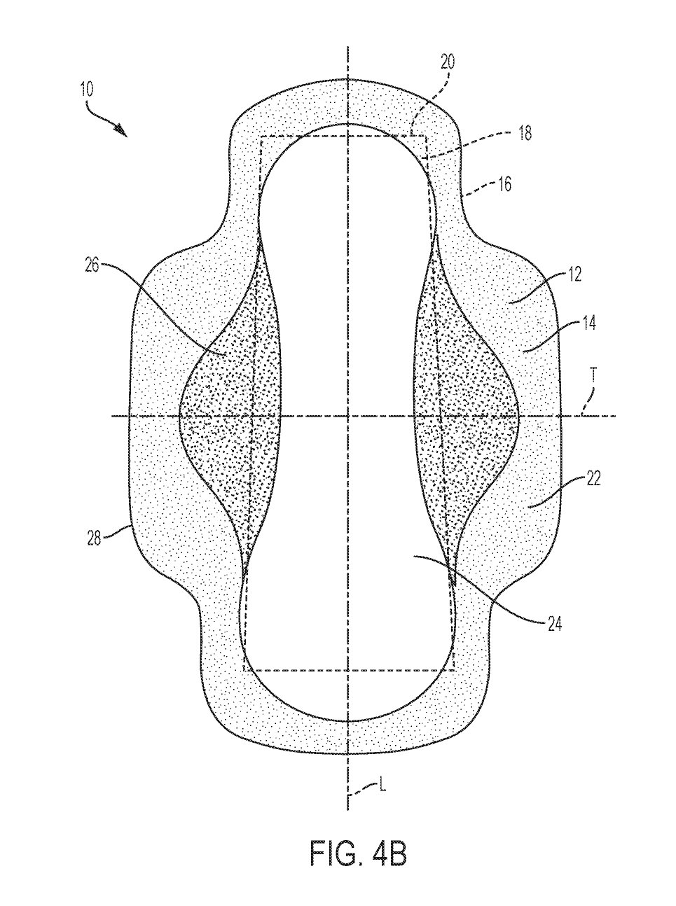

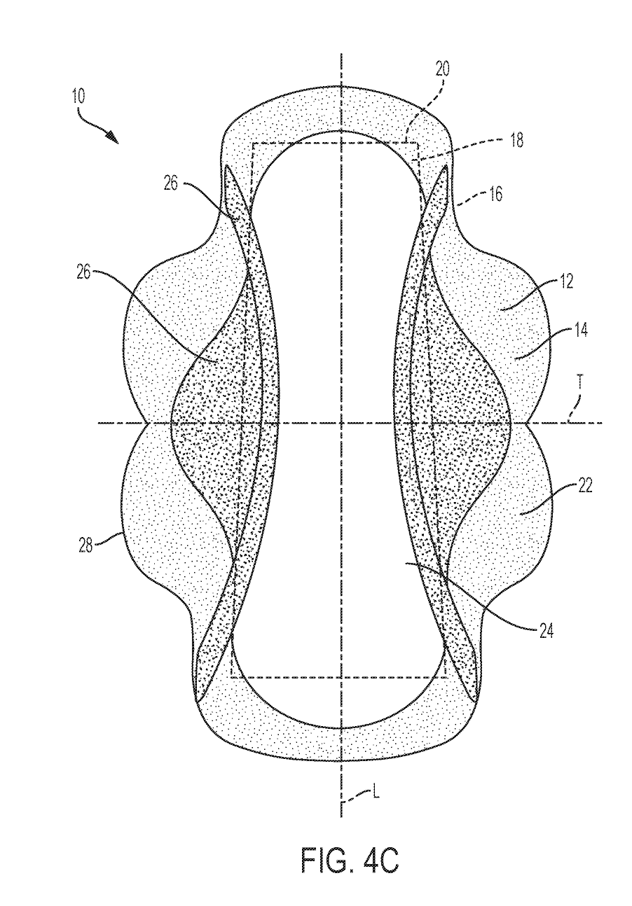

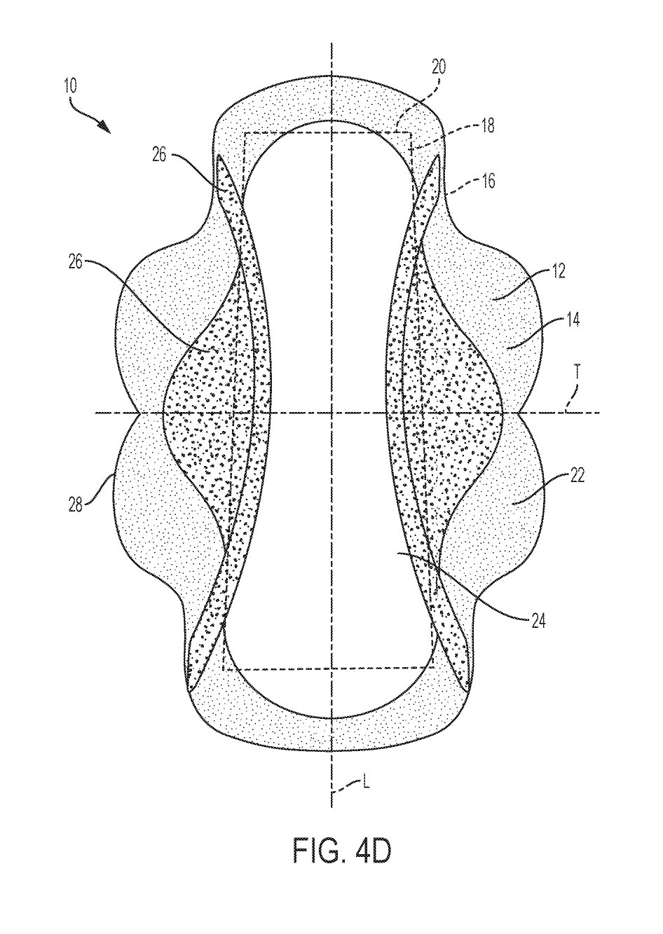

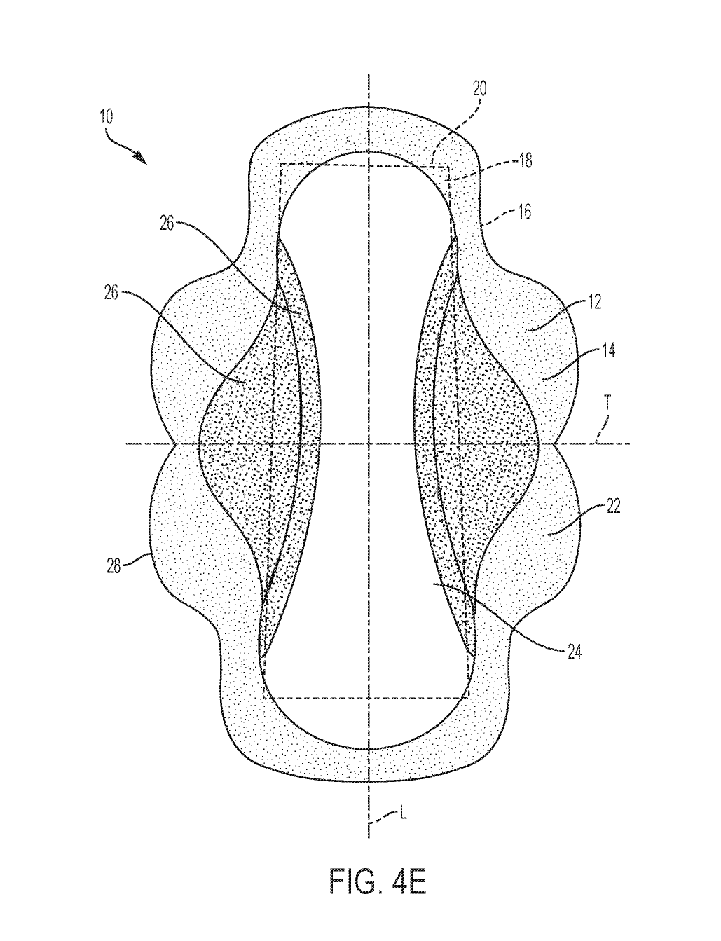

FIGS. 4A-4E show an absorbent article 10, e.g., having a body-contacting surface 12 comprising an outer surface of a topsheet 14, a liquid impervious backsheet 16 joined to the topsheet 14, and an absorbent core 18. The absorbent article 10 has a longitudinal axis L, a transverse axis T. The longitudinal axis and the transverse axis meet at a central point of the sanitary napkin. The product graphic framing arrangement 22 frames the central usage area 24 of the absorbent core 18. As shown in FIGS. 4A-4E, the product graphic framing arrangement may incorporate additional graphical elements 26. The additional graphical elements 26 may be part of the product graphical framing arrangement 22 as shown in FIGS. 4C-4E or may extend from the product graphical framing arrangement 22 as shown in FIGS. 4A and 4B. As shown in FIG. 4A and 4B, the absorbent product 10 may have different wing designs 28. Additionally, as shown in 4C and 4D, different graphical elements 26 may be different colors or different hues of a color. As shown in FIG. 4E, the graphical elements 26 may run along the frame of the central usage area for a portion of the central usage area.

As shown in FIG. 4A-4E, the absorbent article 10 may include one or more graphical elements to frame the central usage area of the absorbent core 18. Graphical elements 26 can be printed on the body facing surface or the garment facing surface of the topsheet 14 so that they are readily visible. Additional graphical elements 26 can be printed on any layer of the absorbent article 10. Additional graphical elements 26 can be printed on the body facing side of the backsheet 16, on the body facing side of a secondary topsheet (not shown), on a body facing side of an absorbent core 18, or on a portion of a body facing side of the absorbent core 18.

The graphical elements can have different sizes, colors, shapes, and designs. The graphical elements can be balanced to the extent that several smaller graphical elements on one side can be “balanced” by a large graphical element on the other side, or smaller objects can be placed further away from the center than larger objects. Alternatively, a darker object can be similarly balanced by several lighter objects. It also should be noted that although the graphical elements are shown herein as possessing relatively simple shapes, this is merely for exemplary purposes. Virtually any shape or design can be employed, such as dots, ovals, triangles, squares, rectangles, flowers, butterflies, stars, hearts, spirals, double spirals, clothold curves, comu spirals, polynomial double spirals, Euler's double spirals, parametric double spirals, etc. In fact, it is generally preferable that the graphical elements are more complicated in nature and that they possess some degree of curvature and color to provide a more complex visual perception and aid in the overall masking effect of the article.

In certain cases, the graphical elements can also be arranged to impart an optical illusion when viewed from a particular focal point of the user. For instance, the arrangement of the graphical elements relative to one another can form an optical illusion of motion perceivable as at least one of the objects appearing to move away from an edge of the body. Exemplary objects for providing such an illusionary pattern can include geometric, spiral, anomalous motion, rotational, color change, peripheral drift, positive after image blurs, scintillation grid, stereopsis and chromosteropsis, contraction and expansion, contrast polarities, convection, chromatic assimilation, etc., such as described in U.S. Patent Publication Nos. 2009/0157021 to Sullivan, et al. and 2005/0268371 to Meekins, which are incorporated herein in their entirety by reference thereto for all purposes. In many embodiments, graphical elements of different shades can be employed to create a perception of depth within the absorbent article by a user looking upon the viewing surface of the topsheet. Such illusionary patterns are described in U.S. Patent Publication No. 2003/0114811 to Christen, et al., which is incorporated herein in its entirety by reference thereto for all purposes.

The product graphic framing arrangement may use one or more graphics to form a frame around the central functional area of the absorbent core such that the graphics occupy 80% or more of the core perimeter. The graphics may be connected. When using non-identical cores, between 0.5% and 20% of the absorbent core may be located outside of the frame formed by the one or more connected graphics, such as, for example, between 1% and 15%, between 4% and 10%, between 5% and 9%.

The product graphic framing arrangement highlights the central usage area of the absorbent core of the article while allowing for flexibility during high speed manufacturing. The inventors have found that by balancing the location of the graphical elements and by placing graphical elements along the absorbent core perimeter that highlight the central usage area of the absorbent core, one can achieve a product graphic orientation that allows for high speed manufacturing while being consumer acceptable. The graphic orientation may be used with identical cores or with non-identical cores.

Additional graphical elements 26 may be used in conjunction with the product graphic framing arrangement. The product graphic framing arrangement may incorporate the additional graphical elements. The additional graphical elements may be used to contrast with the product graphic framing arrangement by using different colors, hue angles, luminosities, or color intensities.

In many embodiments, the functional area can have an additional functional indicator within the perimeter of the absorbent core. The additional functional indicator can be one or more additional graphical elements, apertures, perforations, slots, or dots in the core. The additional functional indicator can be located under the topsheet, in or on the absorbent core, or any other layer that is visible from the body facing surface of the topsheet.

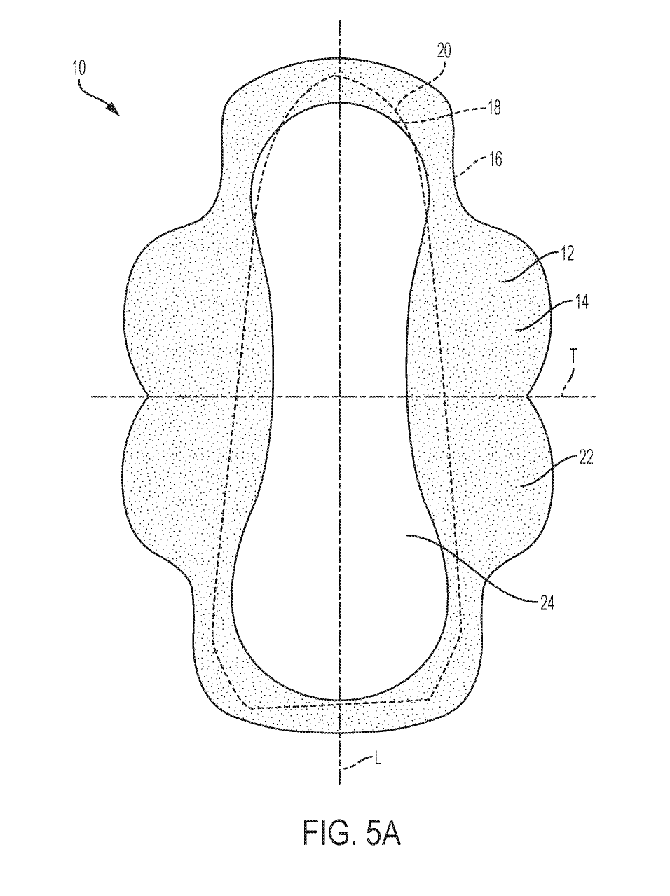

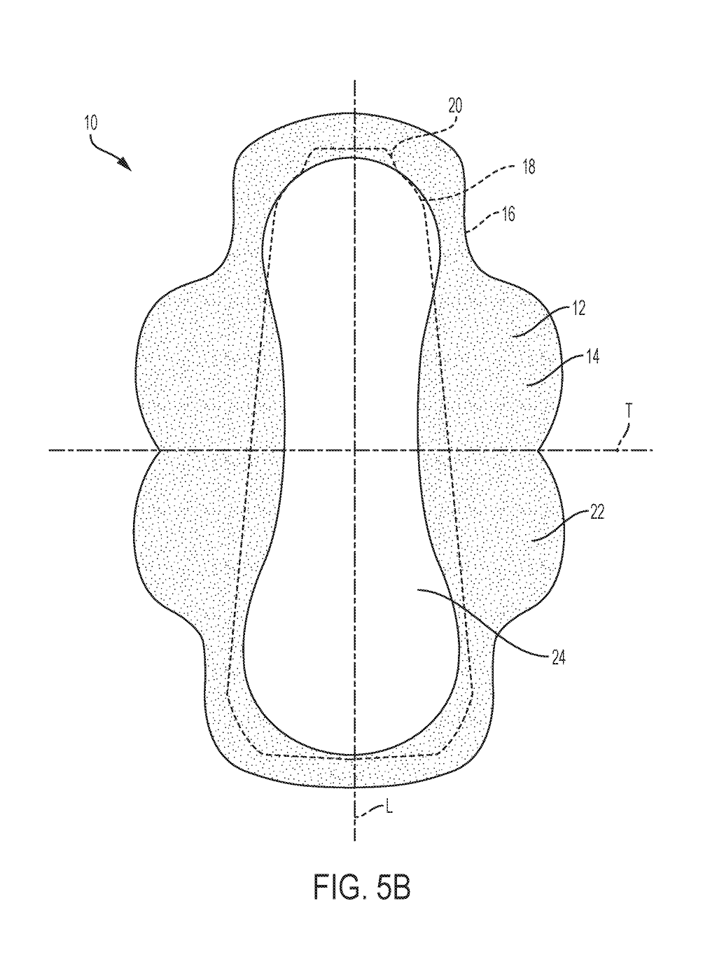

FIGS. 5A and 5B represent absorbent articles 10, e.g., having a body-contacting surface 12 comprising an outer surface of a topsheet 14, a liquid impervious backsheet 16 joined to the topsheet 14, and an absorbent core 18. The absorbent cores 18 of 5A and 5B are non-identical. The absorbent article 10 has a longitudinal axis L, a transverse axis T. The longitudinal axis and the transverse axis meet at a central point of the sanitary napkin. The product graphic framing arrangement 22 frames the central usage area 24 of the absorbent core 18. As shown in FIG. 5A and FIG. 5B, the use of the product graphic framing arrangement 22 allows for the variability created by using non-identical cores without sacrificing consumer acceptance. The product graphic framing arrangement 22 serves to highlight the central usage area 24 of the absorbent core 18 while drawing attention away from the variability created by using non-identical cores in high speed manufacturing.

The absorbent article can also be provided with additional features commonly found in napkins, including “wings” or “flaps” as is known in the art, and/or a secondary topsheet, and/or a fluid acquisition layer, and/or other layers designed to promote fluid transport to absorbent core 18. Likewise, topsheet 14 of the sanitary napkin can have various optional characteristics, as is known in the art. For example, topsheet 14 can have one or more of channels embossed therein to direct fluid flow, apertures therethrough to aid in fluid acquisition, and printed signals visible on or through, the visible signals being printed on the topsheet or underlying layers for functional and esthetic properties.

The topsheet can be manufactured from a wide range of materials such as woven and nonwoven materials; polymeric materials such as apertured formed thermoplastic films, apertured plastic films, and hydroformed thermoplastic films; porous foams; reticulated foams; reticulated thermoplastic films; and thermoplastic scrims. Suitable woven and nonwoven materials can be included of natural fibers (e.g., wood or cotton fibers), synthetic fibers (e.g., polymeric fibers such as polyester, polypropylene, or polyethylene fibers) or from a combination of natural and synthetic fibers. In many embodiments, the topsheet can be made of a hydrophobic material to isolate the wearer's skin from liquids which have passed through the topsheet. If the topsheet is made of a hydrophobic material, at least the upper surface of the topsheet is treated to be hydrophilic so that liquids will transfer through the topsheet more rapidly. This diminishes the likelihood that body exudates will flow off the topsheet rather than being drawn through the topsheet and being absorbed by the absorbent core. In many embodiments, the topsheet can be rendered hydrophilic by treating it with a surfactant. Suitable methods for treating the topsheet with a surfactant include spraying the topsheet material with the surfactant and immersing the material into the surfactant.

The topsheet can include an apertured formed film. Apertured formed films can be used for the topsheet because they are pervious to body exudates and yet non-absorbent and have a reduced tendency to allow liquids to pass back through and rewet the wearer's skin. Thus, the surface of the formed film which is in contact with the body remains dry, thereby reducing body soiling and creating a more comfortable feel for the wearer. Suitable formed films are described in U.S. Pat. No. 3,929,135, entitled “Absorptive Structures Having Tapered Capillaries”, issued to Thompson on Dec. 30, 1975; U.S. Pat. No. 4,324,246 entitled “Disposable Absorbent Article Having A Stain Resistant Topsheet”, issued to Mullane, et al. on Apr. 13, 1982; U.S. Pat. No. 4,342,314 entitled “Resilient Plastic Web Exhibiting Fiber-Like Properties”, issued to Radel, et al. on Aug. 3, 1982; U.S. Pat. No. 4,463,045 entitled “Macroscopically Expanded Three-Dimensional Plastic Web Exhibiting Non-Glossy Visible Surface and Cloth-Like Tactile Impression”, issued to Ahr, et al. on Jul. 31, 1984; and U.S. Pat. No. 5,006,394 “Multilayer Polymeric Film” issued to Baird on Apr. 9, 1991.

The absorbent core can be any absorbent member which is generally compressible, conformable, non-irritating to the wearer's skin, and capable of absorbing and retaining body fluids. The absorbent core can be manufactured in a wide variety of sizes and shapes (e.g., rectangular, hourglass, “T” -shaped, asymmetric, etc.) and from a wide variety of liquid-absorbent materials commonly used in disposable pull-on garments and other absorbent articles such as comminuted wood pulp which is generally referred to as airfelt. Examples of other suitable absorbent materials include creped cellulose wadding; meltblown polymers including coform; chemically stiffened, modified or cross-linked cellulosic fibers; tissue including tissue wraps and tissue laminates; absorbent foams; absorbent sponges; superabsorbent polymers; absorbent gelling materials; or any equivalent material or combinations of materials.

The configuration and construction of the absorbent core can vary (e.g., the absorbent core can have varying caliper zones, a hydrophilic gradient, a superabsorbent gradient, or lower average density and lower average basis weight acquisition zones; or can include one or more layers or structures). Further, the size and absorbent capacity of the absorbent core can also be varied to accommodate wearers ranging from infants through adults. However, the total absorbent capacity of the absorbent core should be compatible with the design loading and the intended use of the absorbent article.

The absorbent core can include other optional components. One such optional component is the core wrap, i.e., a material, typically but not always a nonwoven material, which either partially or totally surrounds the core. Suitable core wrap materials include, but are not limited to, cellulose, hydrophilically modified nonwoven materials, perforated films and combinations thereof.

The backsheet can comprise a liquid impervious film. The backsheet can be impervious to liquids (e.g., body fluids) and can be typically manufactured from a thin plastic film. However, typically the backsheet can permit vapours to escape from the disposable article. In many embodiments, a microporous polyethylene film can be used for the backsheet. A suitable microporous polyethylene film is manufactured by Mitsui Toatsu Chemicals, Inc., Nagoya, Japan and marketed in the trade as PG-P.

One suitable material for the backsheet can be a liquid impervious thermoplastic film having a thickness of from about 0.012 mm (0.50 mil) to about 0.051 mm (2.0 mils), for example including polyethylene or polypropylene. Typically, the backsheet can have a basis weight of from about 5 g/m² to about 35 g/m². However, it should be noted that other flexible liquid impervious materials can be used as the backsheet. Herein, “flexible” refers to materials which are compliant and which will readily conform to the general shape and contours of the wearer's body.

The backsheet can be typically positioned adjacent the outer-facing surface of the absorbent core and can be joined thereto by any suitable attachment device known in the art. For example, the backsheet can be secured to the absorbent core by a uniform continuous layer of adhesive, a patterned layer of adhesive, or an array of separate lines, spirals, or spots of adhesive. Illustrative, but nonlimiting adhesives, include adhesives manufactured by H. B. Fuller Company of St. Paul, Minn., U.S.A., and marketed as HL-1358J. An example of a suitable attachment device including an open pattern network of filaments of adhesive is disclosed in U.S. Pat. No. 4,573,986 entitled “Disposable Waste-Containment Garment”, which issued to Minetola et al. on Mar. 4, 1986. Another suitable attachment device including several lines of adhesive filaments swirled into a spiral pattern is illustrated by the apparatus and methods shown in U.S. Pat. No. 3,911,173 issued to Sprague, Jr. on Oct. 7, 1975; U.S. Pat. No. 4,785,996 issued to Ziecker, et al. on Nov. 22, 1978; and U.S. Pat. No. 4,842,666 issued to Werenicz on Jun. 27, 1989. Alternatively, the attachment device can include heat bonds, pressure bonds, ultrasonic bonds, dynamic mechanical bonds, or any other suitable attachment device or combinations of these attachment devices.

The backsheet can be additionally secured to the topsheet by any of the above-cited attachment devices.

A. An absorbent article comprising a liquid pervious topsheet, the topsheet having an inner surface oriented toward the interior of the absorbent article and an outer surface oriented toward the skin of the wearer when the absorbent article is being worn; a backsheet joined to the topsheet, the backsheet having an inner surface oriented toward the interior of the absorbent article and an outer surface oriented toward the garment of the wearer when the absorbent article is being worn; an absorbent core having a perimeter disposed between the topsheet and the backsheet, the absorbent core having an inner surface oriented toward the skin of the wearer when the absorbent article is being worn and an outer surface oriented toward the garment of the wearer when the absorbent article is being worn; wherein the absorbent article comprises a product graphic framing arrangement; and wherein the product graphic framing arrangement frames the central usage area of the absorbent core.

B. The absorbent article according to paragraph A, wherein the product graphic framing arrangement incorporates one or more graphical elements.

C. The absorbent article according to paragraphs A or B, wherein the product graphic framing arrangement does not extend to an edge of the absorbent article.

D. The absorbent article according to paragraph C, wherein the product graphic framing arrangement extends between 0.5% and 80% of the distance between the central usage area of the absorbent core and the edge of the absorbent article.

E. The absorbent article according to any one of paragraphs A-D, wherein the absorbent core is a non-identical core.

F. The absorbent article according to any one of paragraphs A-E, wherein the product graphic framing arrangement is printed on a garment facing side of the topsheet.

G. The absorbent article according to any one of paragraphs A-F, wherein a functional layer signal is located within the absorbent core perimeter.

H. The absorbent article according to paragraph G, wherein the functional layer signal is selected from the group consisting of a graphical element and a structural identifier.

An array of absorbent articles, the array including the absorbent article according to any one of the paragraphs A-H.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Values disclosed herein as ends of ranges are not to be understood as being strictly limited to the exact numerical values recited. Instead, unless otherwise specified, each numerical range is intended to mean both the recited values and any integers within the range. For example, a range disclosed as “1 to 10” is intended to mean “1, 2, 3, 4, 5, 6, 7, 8, 9, and 10.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed:

1. An absorbent article comprising:
a liquid pervious topsheet, the topsheet having an inner surface oriented toward the interior of the absorbent article and an outer surface oriented toward the skin of the wearer when the absorbent article is being worn;
a backsheet joined to the topsheet, the backsheet having an inner surface oriented toward the interior of the absorbent article and an outer surface oriented toward the garment of the wearer when the absorbent article is being worn;
an absorbent core having a perimeter disposed between the topsheet and the backsheet, the absorbent core having an inner surface oriented toward the skin of the wearer when the absorbent article is being worn and an outer surface oriented toward the garment of the wearer when the absorbent article is being worn;
wherein the absorbent article comprises a product graphic framing arrangement; and
wherein the product graphic framing arrangement frames the central usage area of the absorbent core.

2. The absorbent article of claim 1, wherein the product graphic framing arrangement incorporates one or more graphical elements.

3. The absorbent article of claim 1, wherein the product graphic framing arrangement does not extend to an edge of the absorbent article.

4. The absorbent article of claim 3, wherein the product graphic framing arrangement extends between 0.5% and 80% of the distance between the central usage area of the absorbent core and the edge of the absorbent article.

5. The absorbent article of claim 1, wherein the absorbent core is a non-identical core.

6. The absorbent article of claim 1, wherein the product graphic framing arrangement is printed on a garment facing side of the topsheet.

7. The absorbent article of claim 1, wherein a functional layer signal is located within the absorbent core perimeter.

8. The absorbent article of claim 7, wherein the functional layer signal is selected from the group consisting of a graphical element and a structural identifier.

9. An array of absorbent articles, each absorbent article comprising:
a liquid pervious topsheet, the topsheet having an inner surface oriented toward the interior of the absorbent article and an outer surface oriented toward the skin of the wearer when the absorbent article is being worn;
a backsheet joined to the topsheet, the backsheet having an inner surface oriented toward the interior of the absorbent article and an outer surface oriented toward the garment of the wearer when the absorbent article is being worn;
a non-identical absorbent core having a perimeter disposed between the topsheet and the backsheet, the absorbent core having an inner surface oriented toward the skin of the wearer when the absorbent article is being worn and an outer surface oriented toward the garment of the wearer when the absorbent article is being worn;
a functional layer signal generally centrally located on a layer beneath the topsheet;
wherein the absorbent product comprises a product graphic framing arrangement visible from the body facing surface.

10. The array of absorbent articles of claim 9, wherein the product graphic framing arrangement incorporates one or more graphical elements.

11. The array of absorbent articles of claim 9, wherein the product graphic framing arrangement does not extend to an edge of the absorbent article.

12. The array of absorbent articles of claim 11, wherein the product graphic framing arrangement extends between 0.5% and 80% of the distance between the central usage area of the absorbent core and the edge of the absorbent article.

13. The array of absorbent articles of claim 9, wherein the product graphic framing arrangement is printed on a garment facing side of the topsheet.

14. The array of absorbent articles of claim 9, wherein a functional layer signal is located within the absorbent core perimeter.
